(19) United States
(12) Patent Application Publication
(10) Pub. No.: US 2010/0092956 A1
(43) Pub. Date: Apr. 15, 2010

(54) Analytical Processing and Detection Device

(75) Inventors: Roman Egli, Zurich (CH); Alan Furlan, Immensee (CH); Sascha Roehrig, Lucern (CH)

Correspondence Address: Roche Molecular Systems, Inc.;Patent Law Department, 4300 Hacienda Drive, Pleasanton, CA, 94588, US

(73) Assignee: ROCHE MOLECULAR SYSTEMS, INC., Pleasanton, CA (US)

(21) Appl. No.: 12/419,871

(22) Filed: Apr. 7, 2009

(30) Foreign Application Priority Data
Apr. 8, 2008 (EP) 08103426.6

(51) Int. Cl. C12Q 1/68; C12M 1/34

(52) U.S. Cl. 435/6; 435/287.2

(57) ABSTRACT

An analytical processing and detection device is described which comprises a rack capable of holding at least one reaction receptacle, at least one magnetic unit for exerting a magnetic field on at least one reaction receptacle comprising magnetic particles in a fluid, wherein said at least one magnetic unit is reversibly or irreversibly connected to said rack, and wherein said magnetic field causes the magnetic particles to be sequestered to the side walls of said at least one reaction receptacle, and a detection unit for detecting a signal in the fluid in said reaction receptacle.

### CROSS-REFERENCE TO RELATED APPLICATION

The present application claims the benefit of EP Appl. No. 08103426.6 filed Apr. 8, 2008, the content of which is hereby incorporated herein by reference in its entirety.

### FIELD OF THE INVENTION

The present invention relates to an analytical processing and device for the detection of a signal in the fluid of an analytic receptacle in the presence of magnetic particles.

### DESCRIPTION OF PRIOR ART

Commonly, for analytical methods based on gene or protein expression or genetic analysis, analytes in biological samples are prepared by adsorption (binding) to a solid phase, such as magnetic particles, and then further processed to obtain a detection signal, thus obtaining an analytical result that can be used in diagnosis of disease or in the monitoring for treatment.

In the field of PCR diagnostics, the analyte which is bound to the particles is washed and may be eluted prior to further processing. The eluate is then transferred to new receptacles for PCR amplification and detection of amplified PCR product in the liquid. In such a setting, no particles are transferred and PCR amplification and detection are carried out in the absence of the particles, which would disturb the optical detection of the detection signal in the liquid. However, the elution and transfer of the analyte has the disadvantage that the analyte comprised in one reaction receptacle can not be completely transferred due to residual liquid remaining in the reaction receptacle (interstitial volume). In the field of PCR diagnostics, where small amounts of sample are processed and analyzed, such incomplete transfer of liquids impairs the sensitivity of the analysis.

WO03/057910 discloses a method of amplifying and optically detecting the detection signal in the liquid phase of receptacles without removal of the magnetic particles. The particles are allowed to settle to the bottom of the receptacles by sedimentation.

### SUMMARY OF THE INVENTION

In one aspect, the invention relates to an analytical processing and detection device comprising:

at least one reaction receptacle having an interior side wall,

a rack comprising at least one cavity capable of receiving said reaction receptacle,

at least one magnetic unit for exerting a magnetic field on said reaction receptacle comprising magnetic particles capable of binding an analyte contained in a fluid, wherein said at least one magnetic unit is coupled to said rack, and wherein said magnetic field generated by at least one magnetic unit causes the magnetic particles to be sequestered to the interior side wall of the reaction receptacle, and

a detection unit for detecting a signal in the fluid in said reaction receptacle, wherein said detection unit is arranged within said device to detect a signal from said reaction receptacle while said reaction receptacle comprising said magnetic particles is held in said rack.

In another aspect, the invention relates to a method for determining the amount of an analyte bound to magnetic particles present in a sample fluid in a reaction receptacle, said method comprising the steps of:

a) providing a biological sample comprising an analyte in at least one reaction receptacle,
b) binding said analyte to magnetic particles in said at least one reaction receptacle,
c) processing said analyte bound to magnetic particles in said at least one reaction receptacle to obtain a detection signal, said detection signal being a signal that is disturbed by the presence of said magnetic particles, wherein said at least one reaction receptacle is located in an analytical processing unit,
d) generating a supernatant by applying a magnetic field on the contents of said at least one reaction receptacle and sequestering said magnetic particles to the interior side wall of said reaction receptacle, and
e) detecting said detection signal corresponding to an analyte concentration in the supernatant of said reaction receptacle in the presence of the sequestered magnetic particles, wherein steps c) to e) are carried out in the same analytical processing unit.

### BRIEF DESCRIPTION OF THE FIGURES

FIG. 1 shows an image of emitted fluorescent light from wells of a 96 well plate with different amounts of magnetic particles.

FIG. 2 shows the effects of different degrees of sequestration of the magnetic glass particles.

FIG. 3 shows schematic views of a certain arrangement of the magnets in the heat block.

FIG. 4 shows a possible arrangement of magnets and cavities.

FIG. 5 shows a device comprising a detection unit, a rack holding a receptacle and magnets coupled to said rack.

### DETAILED DESCRIPTION OF THE INVENTION

As described above, the device according to the invention can be used in a method for processing the contents of at least one reaction receptacle and determining the amount of an analyte bound to magnetic particles present in each of said reaction receptacles, wherein said analyte can be processed and determined in the presence of the magnetic particles accurately and with high sensitivity by connecting one or more magnetic units to said rack. The magnetic unit(s) is/are coupled to the rack such that the magnetic particles are sequestered on the side walls of the reaction receptacles. By sequestering the magnetic particles to the side wall, the liquid in the well becomes essentially free of magnetic particles which can interfere with the detection method. In a certain embodiment, the center of the well is free of magnetic particles. The term “coupled” as used herein relates to any type of connection between magnet and rack. The magnet(s) may be reversibly coupled to said rack. The magnets may be stably integrated into the rack.

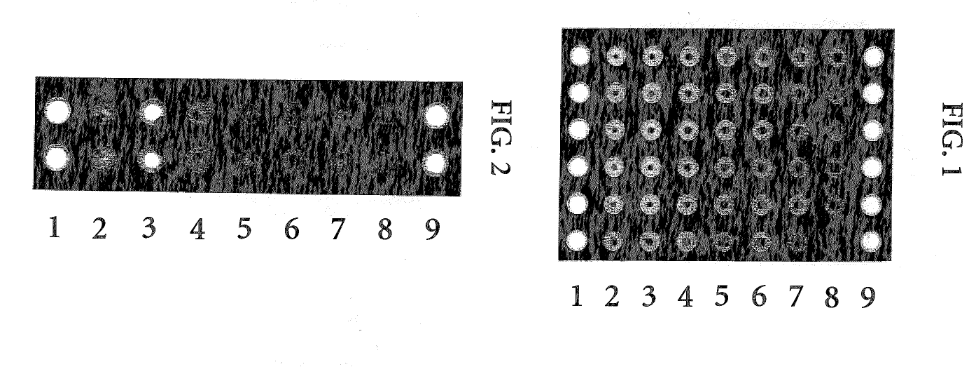

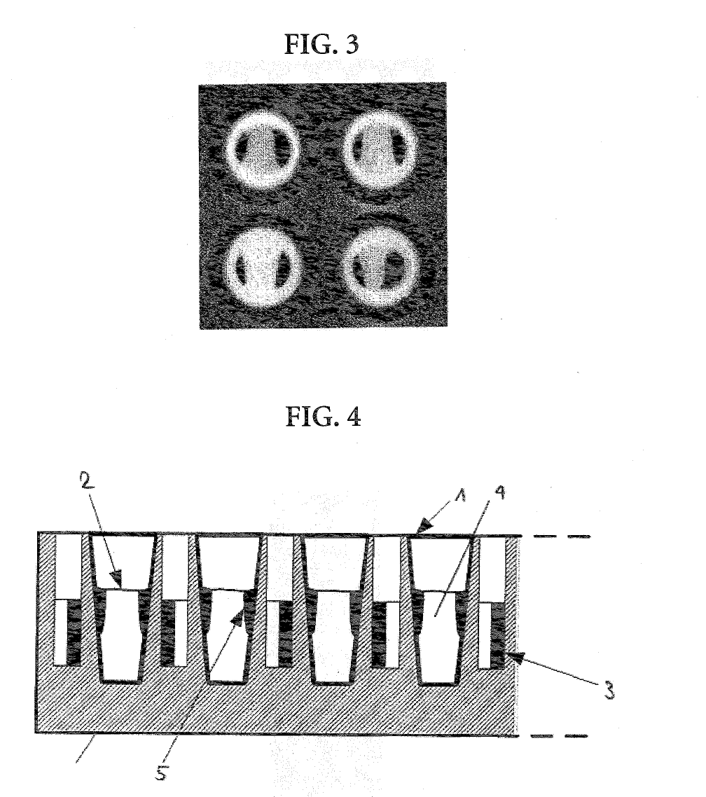

Thus, the analyte can be processed without need for separating the eluate from the magnetic particles, because the detection signal is only minimally impaired by the magnetic particles (see FIG. 3). The term “separating the eluate from the magnetic particles” means that the eluate is physically or chemically separated, and that following separation, is no longer in physical contact with from the magnetic particles. FIG. 1 shows how the detection signal is impaired by the presence of unsequestered or insufficiently sequestered magnetic particles. Due to magnetic sequestration of the magnetic particles in the receptacle, the loss of material during pipetting of the eluate due to interstitial volume can be avoided. In addition, although the magnetic particles are not removed, the sequestration to the sidewalls of the reaction receptacles increases of the signal-to-noise ratio caused by suspended or sedimented particles and, thus, improves the sensitivity of the detection.

The term “reaction receptacle” as used herein relates to a receptacle which can hold a liquid and in which a reaction, such as a chemical or enzymatic reaction can take place. Said reaction receptacle comprises a top part having an opening, side walls and a bottom part. The openings may be sealable, e.g. by a cap or a foil. In a certain embodiment, a microtiter plate comprising a plurality of reaction receptacles is employed, for example a 96 well microtiter plate. Shapes of microtiter plates are commonly known. In a certain embodiment, the microtiter plate is white. The white plate improves the efficiency of fluorescence excitation and detection by allowing multiple light reflexion within the beam. The white plates are typically polypropylene plates comprising titanium dioxide as a white color agent. It is important that said plates lack any trace substances which may contribute to plate auto-fluorescence. In certain embodiments, the shape of the reaction receptacles is determined by both thermal and liquid handling considerations.

From the thermal perspective, the shape should be such as to maximize direct thermal contact with the thermal block, in order to increase temperature ramp speed and thermal equilibrium. In certain embodiments, the shape may be a high, thin cylinder or a very short, flat cylinder. In a certain embodiment, the walls of the receptacle have a sufficient inclination angle from vertical such that the microtiter plate can be easily removed from the thermocycler after the PCR amplification. Furthermore, in a certain embodiment the shape of the receptacle is wide enough and has a round bottom to avoid bubble formation during liquid dispensing.

The term “magnetic particles” relates to particles comprising a magnetic material which can bind an analyte in a fluid. Said term encompasses magnetic particles with or without a silica surface. Said magnetic particles are for example magnetic glass particles.

In a certain embodiment, if the analyte is a nucleic acid, said magnetic glass particles are magnetic glass particles comprising an unmodified silica surface. Suitable magnetic glass particles are disclosed in WO 96/41811. The magnetic glass particles are a solid dispersion of small magnetic cores in glass, i.e. they are glass droplets in which very small magnetic objects are dispersed. Those objects that are referred to as magnetic are drawn to a magnet, i.e. ferri- or ferromagnetic or superparamagnetic materials for instance. Paramagnetic substances are not useful as they are only drawn to a magnet very weakly, which is not sufficient for a method according to this invention. Suitable materials are ferri- or ferromagnetic materials, in particular if they have not yet been premagnetized. Premagnetization in this context is understood to mean increasing the remanence by bringing in contact with a magnet. Examples of suitable magnetic materials are iron or iron oxide as e.g. magnetite (Fe₃O₄) or Fe₂O₃, and gamma-Fe₂O₃. In principle, barium ferrite, nickel, cobalt, Al—Ni—Fe—Co alloys or other ferri- or ferromagnetic material could be used. In a certain embodiment according to the present invention magnetic glass particles can be selected from those described in WO96/41811, WO00/32762 and WO98/12717.

In a certain embodiment of the invention, the magnetic glass particles with an unmodified glass surface have a low iron leach. This feature is suitable for the method according to the invention when using magnetic glass particles, as iron is an inhibitor of the subsequent amplification reaction, i.e. iron is an enzymatic inhibitor. This is an important feature of the magnetic glass particles with an unmodified glass surface. In another embodiment of the invention, the magnetic glass particles with an unmodified surface are those described in the European application EP 20 00110165.8 which are also publicly available in the MagNA Pure LC DNA Isolation Kit I (Roche, Mannheim, Germany). The production thereof is summarized below.

Further suitable magnetic glass particles according to the invention are manufactured according to the international application EP1154443 which are also provided in the MagNA Pure LC DNA Isolation Kit I (Roche, Mannheim, Germany)). They are also produced by the sol-gel-method as described in the international application (EP1154443) using magnetic objects or pigments with a diameter of about 23 nm (manufactured by CERAC consisting of γ-Fe2O3; CERAC: P.O. Box 1178, Milwaukee, Wis. 53201-1178 USA; Article-No. I-2012).

According to the present invention, an “analyte” is understood to be any molecule, or aggregate of molecules, including a cell or a cellular component of a virus, found in a sample. Thus, as a non-limiting example, an analyte may be a nucleic acid of interest or a protein of interest which is investigated and its presence or absence, or its concentration in a biological sample is determined as its presence or absence is indicative of a certain condition or disease of a human or animal. Further included in the scope of the term “analyte” are fragments of any such molecule found in a sample. In a certain embodiment, said analyte is a biological analyte, for example a nucleic acid. Said nucleic acid may be RNA or DNA or any derivative thereof. In a certain embodiment, said analyte is a virus, such as the hepatitis A virus (HAV), hepatitis B virus (HBV), hepatitis C virus (HCV), the human immunodeficiency virus (HIV), the human papilloma virus (HPV), parvovirus Bl9, CT/NG. The analyte may also be a bacteria such as mycobacterium avium intracellulare (MAI) or mycobacterium tuberculosis (MTB).

The term “fluid” relates to any kind of liquid comprising said magnetic particles capable of binding an analyte. This may be a biological sample fluid as described hereinafter, or a buffered solution. In one embodiment, said buffered solution is an elution buffer. Elution buffers with a low salt content are for example buffers with a salt content of less than 0.2 mol/l. In a certain embodiment, the elution buffer contains the substance Tris for buffering purposes, for example a Tris buffered solution with a pH around 7 or above 7. In another embodiment, the elution buffer is demineralized water.

The term “rack” as used herein refers to a unit with cavities which can hold at least one reaction receptacle. In a certain embodiment, said rack is a heat conductive rack. This means that the material of said rack has for example a high thermal diffusivity, which is suitable for fast thermal ramps. This is given at high thermal conductivity, for example more than 200 W/(mK), or at least 221 W/(mK), low heat capacity such as less than 1000 J/(kg K), for example equal or less than 900 J/(kg K), and low density, such as less than 12 kg/dm³, for example equal or less than 10.5 kg/dm³. In a certain embodiment, the material of the rack hereinbefore described comprises aluminum or silver. The thermal conductivity of silver is 429 W/(mK), of aluminum 221 W/(mK). The heat capacity of silver is 232 J/(kg K), of aluminum 900 J/(kg K). The density of silver is 10.5 kg/dm³, of aluminum 2.7 kg/dm³. In a certain embodiment, the rack hereinbefore described is a heating block in a thermal cycler. Said heating block is controlled to change between at least two temperatures, for example between an annealing, an incubation and a denaturation temperature used in a polymerase chain reaction, in one cycle. The temperature of said heating block can be changed rapidly. Cooling may be achieved by contacting said heating block with a heat sink, e.g. a liquid. Or it may be achieved by a thermoelectric element, such as a Peltier element. A combination of a resistive heater and a thermoelectric cooling element, or a thermoelectric element used in both heating and cooling mode are the most frequently used configurations, apart from heating and cooling by using air.

The term “magnetic unit” as used herein relates to any unit which can exert a magnetic field. Said magnetic unit may be an electromagnetic unit or a permanent magnet.

In an embodiment, the magnetic field exerted on the reaction receptacles comprising said magnetic particles as described hereinbefore can be increased or decreased. Such increases of the magnetic field may be achieved by moving a magnet into proximity with the rack holding the reaction receptacles. Consequently, decreases may be achieved for example by removing said magnet from the rack.

In another embodiment of the invention hereinbefore described, said one or more units for exerting a magnetic field on at least one reaction receptacle comprising magnetic particles in the fluid are one or more permanent magnets which are stably integrated into said rack. In a certain embodiment, said magnetic units are at least two magnetic units. In a certain embodiment, said one or more permanent magnets are thermostable at temperatures of up to 140° C. This means that ability of the permanent magnet to generate a magnetic field is not impaired by such high temperatures. This allows the generation of a magnetic field even during thermocycling, which necessitates heating of the contents of the liquid inside the reaction receptacle to temperatures as high as 100° C. The thermal block itself can transiently heat up to temperatures of 110° C. This so-called block overshoot is used to accelerate the equilibration of the liquid to the final temperature. In a certain embodiment, said one or more permanent magnets are thermostable at temperatures of up to 180° C. In another embodiment, said one or more permanent magnets are thermostable at temperatures of up to 250° C. In a another embodiment, said one or more permanent magnets comprise a SmCo alloy.

The geometry and placement of the permanent magnet hereinbefore described may also affect the efficiency of sequestration of the magnetic particles to the interior side walls of the reaction receptacles, as hereinbefore described. Therefore, in a certain embodiment, said cavities for receiving reaction receptacles are arranged between at least two permanent magnets such that equal magnetic poles of said permanent magnets face the same cavities.

In another embodiment of the device hereinbefore described, said two or more permanent magnets are pin-shaped and said magnets extend over the whole width or length of said rack. In another embodiment, said permanent magnets are pin-shaped, wherein the length of said magnets corresponds approximately to the diameter of a cavity of said rack, and wherein said magnet is arranged between two cavities for receiving reaction receptacles of said rack such that equal magnetic poles of said permanent magnets face the same cavity. In another embodiment, the magnetic field is adjusted such that the area of the collected beads projected onto a horizontal surface should be minimal. Typically, the beads are well focused just below the liquid surface.

The device hereinbefore described is suitable for detecting a signal which is impaired by the presence of magnetic particles. The term “detection unit” therefore relates to a detection unit which can detect a signal which is impaired by the presence of magnetic particles. In an embodiment said detection unit is an optical unit. In an embodiment said signal is fluorescence. Said detection units may comprise photodiodes or CCD chips (as described, e.g. in WO99/60381 or DE19748211). Said detection units further comprise a light source for emitting excitation light, such as a 100 watt halogen lamp (WO99/60381) or an array of LEDs. Said detection device my further optionally comprise a beam splitter ((DE10131687, DE10155142). Furthermore, said detection device may comprise Fresnel lenses (U.S. Pat. No. 6,246,525), field lenses (EP 1681556) or one or more telecentric lenses (U.S. Pat. No. 6,498,690).

Further to the device hereinbefore described, the present invention also relates to an analytical system comprising a device as described hereinbefore.

Such analytical system and device can be used for performing a method for determining the amount of an analyte bound to magnetic particles present in a sample fluid in a reaction receptacle, said method comprising the steps of:
a) providing a biological sample comprising an analyte in at least one reaction receptacle,
b) binding said analyte to magnetic particles in said at least one reaction receptacle, and
c) processing said analyte bound to magnetic particles in said at least one reaction receptacle to obtain a detection signal.

The detection signal is a signal that is disturbed by the presence of said magnetic particles. In a certain embodiment, the detection signal is an optical signal. Furthermore, in step c) the at least one reaction receptacle is located in an analytical processing unit.

In step d) a supernatant is generated by applying a magnetic field on the contents of said at least one reaction receptacle. The magnetic field causes the magnetic particles to be sequestered to the interior side wall of the reaction receptacle. Step d) is followed by step e), in which a detection signal corresponding to an analyte concentration in the supernatant of said at least one reaction receptacle is detected, for example by optical detection, in the presence of the sequestered magnetic particles. In the method of the present invention, steps c) to e) are carried out in the same analytical processing unit.

In a certain embodiment of this method all steps are automated. Automated method means that the steps of the automatable method are carried out with an apparatus or machine capable of operating with little or no external control or influence by a human being.

In a certain embodiment of the invention, the method is in a high-throughput format, i.e. the automated method is carried out in a high-throughput format which means that the methods and the used machine or apparatus are optimized for a high-throughput of >100 samples in a short time.

The term “supernatant” as used herein relates to the liquid in a reaction receptacle after sequestration of magnetic particles by a magnetic field. Thus, the supernatant refers to the liquid in the reaction receptacles after the magnetic particles were sequestered by the magnetic field, while the magnetic particles form a pellet at the site of the side walls of the reaction receptacles to which they were sequestered.

The term “biological sample” as used herein relates to any sample derived from a biological organism. In an embodiment of the invention, the biological sample comprises viruses or bacterial cells, as well as isolated cells from multicellular organisms as e.g. human and animal cells such as leucocytes, and immunologically active low and high molecular chemical compounds such as haptens, antigens, antibodies and nucleic acids, blood plasma, cerebral fluid, sputum, stool, biopsy specimens, bone marrow, oral rinses, blood serum, tissues, urine or mixtures thereof. Thus, the biological sample may be either solid or fluid. In a certain embodiment of the invention the biological sample is a fluid from the human or animal body. A biological sample which is a fluid is also called a sample fluid. The biological sample may be blood, blood plasma, blood serum or urine. The blood plasma is for example a EDTA-, heparin- or citrate-treated blood plasma. In an embodiment of the invention the biological sample comprises bacterial cells, eukaryotic cells, viruses or mixtures thereof. In a certain embodiment of the invention, the virus is the hepatitis A virus (HAV), hepatitis B virus (HBV), hepatitis C virus (HCV), the human immunodeficiency virus (HIV), the human papilloma virus (HPV), parvovirus B19, CT/NG, CMV, and the bacteria are mycobacterium tuberculosis (MTB) or mycobacterium avium intracellulare (MAI). The biological sample can also be of a type used for environmental analysis, food analysis or molecular biology research, e.g. from bacterial cultures or phage lysates.

The biological sample comprising a mixture of biological compounds comprising non-target and a target nucleic acid need not be lysed, when the biological sample can be used without pretreatment in the method according to the invention. However, a biological sample comprising non-target nucleic acids and a target nucleic acid may be lysed to create a mixture of biological compounds comprising non-target and a target nucleic acid. Therefore, the biological compounds, non-target nucleic acids and the target nucleic acid contained in the biological sample are released, creating a mixture of biological compounds comprising non-target nucleic acids and the target nucleic acid. Procedures for lysing biological samples are known by the person skilled in the art and can be chemical, enzymatic or physical in nature. A combination of these procedures is applicable as well. For instance, lysis can be performed using ultrasound, high pressure, shear forces, alkali, detergents or chaotropic saline solutions, or proteases or lipases. For the lysis procedure to obtain nucleic acids, special reference is made to Sambrook et al.: Molecular Cloning, A Laboratory Manual, 2nd Addition, Cold Spring Harbour Laboratory Press, Cold Spring Harbour, N.Y. and Ausubel et al.: Current Protocols in Molecular Biology 1987, J. Wiley and Sons, NY.

The magnetic glass particles used in the present invention may be provided in different formulations essentially as described in European patent publication EP1154443. It is possible to provide them in the form of a tablet, as a powder or for example as a suspension. In a certain embodiment of the invention these suspensions contain between 5 to 60 mg/ml magnetic glass particles (MGPs). In another embodiment of the invention the silica-containing material is suspended in aqueous buffered solutions which may optionally contain a chaotropic agent in a concentration of between 1 and 8 mol/1, for example between 2 and 8 mol/l, and such as between 2 and 6 mol/l, and more specifically between 4 and 6 mol/l. Chaotropic salts are sodium iodide, sodium perchlorate, guanidinium thiocyanate, guanidinium isothiocyanate or guanidinium hydrochloride. A chaotropic agent according to the present invention is any chemical substance which will disturb the ordered structure of liquid water and will have the effect that DNA or RNA will bind to the MGPs according to the present invention if this agent is present in the DNA or RNA containing solution. Other compounds known to the expert in the field are also possible. The purification effect results from the behavior of DNA or RNA to bind to material with a glass surface under these conditions i.e. in the presence of certain concentrations of a chaotropic agent, higher concentrations of organic solvents or under acidic conditions. The glass beads with an unmodified glass surface are added to the mixture and incubated for a period of time sufficient for the binding to occur. One of ordinary skill in the art would be familiar with the duration of the incubation step. This step can be optimized by determining the quantity of immobilized nucleic acids on the surface at different points in time. Incubation times of between 10 seconds and 30 minutes can be appropriate for nucleic acids.

A procedure for binding a target nucleic acid (and also the non-target nucleic acids) to magnetic glass particles can be described in detail as follows. Other procedures are also known to the skilled person and may be used as well. While the term “analyte” as defined hereinbefore relates to the target nucleic acid, both target and non-target nucleic acids may be bound to the magnetic glass particles. In one embodiment, the target nucleic acids are bound specifically to the magnetic particles. This specific binding may be mediated, as a non-limiting example, by an oligonucleotide with a sequence which is complementary to the target sequence, whereby said oligonucleotide is firmly bound to the magnetic glass particles. The binding of target and/or non-target nucleic acid is for example performed in the presence of chaotropic salts in concentrations as described hereinbefore. Following binding of the analyte to the magnetic particles, the magnetic particles with the bound analyte are separated from the sample. Optionally, the magnetic particles with the bound analyte may be washed with a washing buffer. This may be by separating the material bound to the magnetic particles by applying a magnetic field. For instance, the magnetic particles can be pulled to the wall of the vessel in which incubation was performed. The liquid containing the biological compounds that were not bound to the magnetic particles can then be removed. Therefore, the method according to the invention contains the step of separating said material with said bound non-target nucleic acids and/or said bound target nucleic acid from the non-bound biological compounds.

The removal procedure used depends on the type of vessel in which incubation was performed. Suitable steps include removing the liquid via pipetting or aspiration. The material with the bound DNA or RNA may then be washed at least once, for example with a mixture of 70% v/v ethanol or in an acidic wash solution as described in WO 99/40098. A wash solution is used that does not cause the nucleic acids and the target nucleic acid to be released from the material surface but that washes away the undesired contaminants as thoroughly as possible. This wash step can take place by incubating the magnetic glass particles with the unmodified silica surface with the bound nucleic acids and the target nucleic acid. The material may be resuspended during this step. The contaminated wash solution may be removed just as in the binding step described above. After the last wash step, the material can be dried briefly in a vacuum, or the fluid can be allowed to evaporate. A pretreatment step using acetone may also be performed.

The solution containing the purified analyte is now ready to be further processed. The term “processing” as used herein relates to any manipulation of the analyte, e.g. by chemical or biochemical manipulation, which leads to the production of a detection signal.

In a certain embodiment of the method hereinbefore described, the processing of said nucleic acid comprises amplification. Amplification is a well known method to multiply copies of a specific sequence of RNA or DNA which can then be detected and analyzed quantitatively or qualitatively. In another embodiment, said processing comprises elution of said analyte from the magnetic beads into the fluid in said reaction receptacle. For elution to take place, the material with the unmodified silica surface is resuspended in a solution with no or only a low amount of chaotropic agent and/or organic solvent. Alternatively, the suspension can be diluted with a solution with no or only a low amount of chaotropic agent and/or organic solvent. Buffers of this nature are known from DE 3724442 and Analytical Biochemistry 175 (1988) 196-201. The elution buffers with a low salt content are in particular buffers with a salt content of less than 0.2 mol/l. In another embodiment, the elution buffer contains the substance Tris for buffering purposes, in particular a Tris buffered solution with a pH around 7 or above 7. In another special embodiment, the elusion buffer is demineralized water.

The term “processing unit” relates to a unit in which the analyte can be processed for detection. Such a processing unit may be a thermal cycler. In a certain embodiment, said processing unit is the analytical processing and detection device hereinbefore described. Thus, at least steps c) to e) of the method of the present invention can be carried out in said processing and detection device.

For amplification, all reagents necessary for amplification are added to the solution comprising the analyte. Otherwise, a solution containing all reagents necessary for amplification is added to the suspension of the material with the unmodified silica surface and the target nucleic acid.

In a certain embodiment of the invention, the target nucleic acid is amplified with the polymerase chain reaction (PCR). The amplification method may also be the Ligase Chain Reaction (LCR, Wu and Wallace, Genomics 4 (1989)560-569 and Barany, Proc. Natl. Acad. Sci. USA 88 (1991)189-193); Polymerase Ligase Chain Reaction (Barany, PCR Methods and Applic. 1 (1991)5-16); 20 Gap-LCR (PCT Patent Publication No. WO 90/01069); Repair Chain Reaction (European Patent Publication No. EP 439,182 A2), 3SR (Kwoh, et al., Proc. Natl. Acad. I Sci. USA 86 (1989)1173-1177; Guatelli, et al., Proc. Natl. Acad. Sci. USA 87 (1990)1874-1878; PCT Patent Publication No. WO 92/0880A), and NASBA (U.S. Pat. No. 5,130,238). Further, there are strand displacement amplification (SDA), transcription mediated amplification (TMA), and Qβ-amplification (for a review see e.g. Whelen and Persing, Annul Rev. Microbiol. 50 (1996) 349-373; Abramson and Myers, Current Opinion in Biotechnology 4 (1993)41-47).

A suitable detection signal of the method hereinbefore described is an optical detection signal. Said optical detection signal is for example fluorescence.

The target nucleic acid may be detected by measuring the intensity of fluorescence light during amplification. This method entails the monitoring of real time fluorescence. In an embodiment, a method exploiting simultaneous amplification and detection by measuring the intensity of fluorescent light is the TaqMan method disclosed in WO92/02638 and the corresponding U.S. Pat. No. 5,210,015, U.S. Pat. No. 5,804,375 and U.S. Pat. No. 5,487,972. This method exploits the exonuclease activity of a polymerase to generate a signal. In detail, the target nucleic acid is detected by a process comprising contacting the sample with an oligonucleotide containing a sequence complementary to a region of the target nucleic acid and a labeled oligonucleotide containing a sequence complementary to a second region of the same target nucleic acid strand, but not including the nucleic acid sequence defined by the first oligonucleotide, to create a mixture of duplexes during hybridization conditions, wherein the duplexes comprise the target nucleic acid annealed to the first oligonucleotide and to the labeled oligonucleotide such that the 3′-end of the first oligonucleotide is adjacent to the 5′-end of the labeled oligonucleotide. Then this mixture is treated with a template-dependent nucleic acid polymerase having a 5′ to 3′ nuclease activity under conditions sufficient to permit the 5′ to 3′ nuclease activity of the polymerase to cleave the annealed, labeled oligonucleotide and release labeled fragments. The signal generated by the hydrolysis of the labeled oligonucleotide is detected and/or measured. TaqMan technology eliminates the need for a solid phase bound reaction complex to be formed and made detectable. In more general terms, the amplification and/or detection reaction of the method according to the invention is a homogeneous solution-phase assay. A further method is in the LightCycler™ format (see e.g. U.S. Pat. No. 6,174,670).

The detection methods may include but are not limited to the binding or intercalating of specific dyes as ethidium bromide which intercalates into the double-stranded DNA and changes its fluorescence thereafter. An excitation beam from a light source is directed onto the liquid in said reaction receptacle. The emission of fluorescent light from the liquid following excitation is indicative of a positive signal, and can be detected and quantitated.

In an embodiment different from the embodiment in which said analyte is a nucleic acid, said analyte is a polypeptide. Processing of said polypeptide in step c) may comprise the steps of:
c1) contacting said analyte bound to magnetic particles with a detection molecule, and
c2) eluting said detection molecule bound to said analyte to obtain a detection signal in a fluid in said reaction receptacles.

In another embodiment, said detection molecule is a labeled antibody which can bind said analyte.

For any of the embodiments of the method hereinbefore described, said magnetic field is produced by a magnetic assembly integrated into a thermoblock. Said magnetic assembly integrated into a thermoblock can be any of the embodiments described hereinabove.

The following examples and figures are provided to aid the understanding of the present invention, the true scope of which is set forth in the appended claims. It is understood that modifications can be made in the procedures set forth without departing from the spirit of the invention.

### Examples

FIG. 1 shows an image of the fluorescent light emitted from the wells of a 96 well microtiter plate (not all wells are shown). Each well comprises an identical volume and concentration of fluorochrome (50 μl of 50 nM fluorescein and 50 μl of Tris buffer) and, from left to right, an increasing amount of magnetic particles (Roche Magnetic Bead particles as used for Cobas Taqman™ and Magnapure™ assays). The first and last columns of wells comprise fluorescent dye without particles. It can be seen that the fluorescence intensity is highest in the absence of magnetic particles and decreases with increasing amount of sedimented magnetic particles. From left to right, each well contains 0, 1, 2, 4, 6, 8, 12, 16 and 0 mg of magnetic particles, respectively.

FIG. 2 shows the effects of different degrees of sequestration of the magnetic particles. Fluorescence intensity in column 3 is significantly increased. Furthermore, FIG. 3 shows that increased intensity can be achieved in multiple wells.

FIG. 4 shows a schematic view of a reaction receptacle (1) comprising a liquid (4). A magnetic unit (3) is located below the meniscus of the liquid (2) such that the magnetic particles (5) are sequestered to the side wall of the reaction receptacle. In order to minimize the loss of emitted light collected by a detector which is located above the well, the area of the collected beads projected onto a horizontal surface should be minimal. This can be achieved by adjusting the magnetic field such that the beads are well focused just below the liquid surface. If the beads are pulled above the liquid surface, they tend to generate a bulge which blocks part of the excitation and emission light.

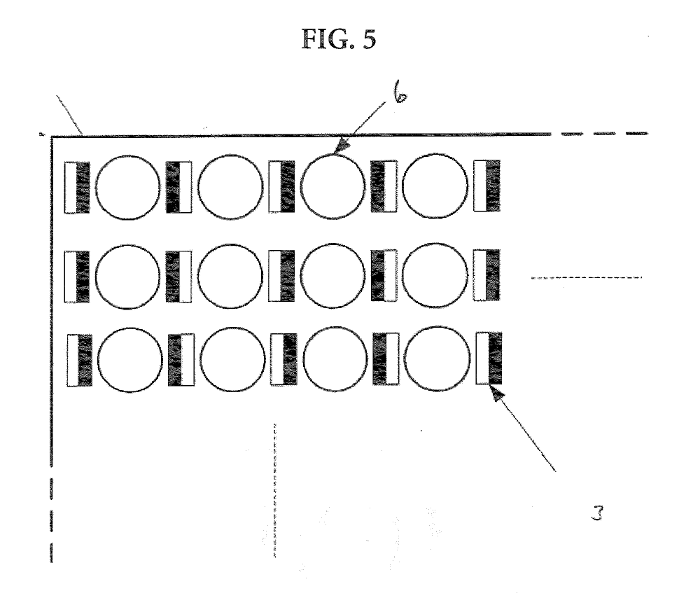

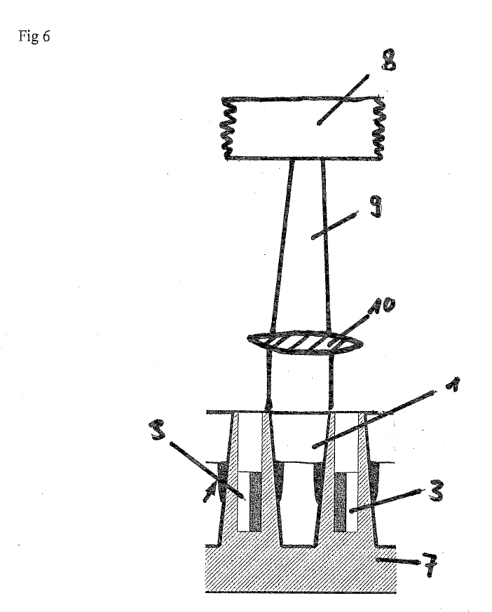

FIG. 5 shows a schematic view of one possible arrangement of cavities (6) and magnetic units (3), whereby the cavities are located between equal magnetic poles (black, white parts) of the magnetic units.

FIG. 6 shows a device with a receptacle (1) which may be an individual receptacle or part of a multiwell plate. Said receptacle is held in a rack (7), and located between equal poles of magnetic units (3). The device also comprises a detection unit (8) which detects the emission light (9). Lenses (10) may be located between the receptacle and the detection device.

While the foregoing invention has been described in some detail for purposes of clarity and understanding, it will be clear to one skilled in the art from a reading of this disclosure that various changes in form and detail can be made without departing from the true scope of the invention. For example, the devices, assemblies and methods described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An analytical processing and detection device comprising:
at least one reaction receptacle having an interior side wall,
a rack comprising at least one cavity capable of receiving said reaction receptacle,
at least one magnetic unit for exerting a magnetic field on said reaction receptacle comprising magnetic particles capable of binding an analyte contained in a fluid, wherein said at least one magnetic unit is coupled to said rack, and wherein said magnetic field generated by at least one magnetic unit causes the magnetic particles to be sequestered to the interior side wall of the receptacle, and
a detection unit for detecting a signal in the fluid in said reaction receptacle, wherein said detection unit is arranged within said device to detect a signal from said reaction receptacle while said reaction receptacle comprising said magnetic particles is held in said rack.

2. The device according to claim 1, wherein said rack is a heat conductive rack.

3. The device according to claim 2, wherein said heat conductive rack is a thermoblock.

4. The device according to claim 1, wherein the material of said rack comprises aluminum or silver.

5. The device according to claim 1, wherein said magnetic field can be increased or decreased.

6. The device according to claim 1, wherein said at least one magnetic unit comprises at least one permanent magnet integrated into said rack.

7. The device according to claim 6, wherein said at least one permanent magnet is thermostable at temperatures of up to about 140° C.

8. The device according to claim 6, wherein said at least one permanent magnet comprises a SmCo alloy.

9. The device according to claim 6, wherein said at least one cavity is arranged between at least two of said permanent magnets such that equal magnetic poles of said permanent magnets face the same cavity.

10. The device according to claim 9, wherein said permanent magnets are located below the meniscus of a liquid comprised in a reaction receptacle which is held by said rack.

11. The device according to claim 1, wherein said detection unit is an optical detection unit.

12. A method for determining the amount of an analyte bound to magnetic particles present in a sample fluid in a reaction receptacle, said method comprising:
a) providing a biological sample comprising an analyte in at least one reaction receptacle,
b) binding said analyte to magnetic particles in said at least one reaction receptacle,
c) processing said analyte bound to magnetic particles in said at least one reaction receptacle to obtain a detection signal, said detection signal being a signal that is disturbed by the presence of said magnetic particles, wherein said at least one reaction receptacle is located in an analytical processing unit,
d) generating a supernatant by applying a magnetic field on the contents of said at least one reaction receptacle and sequestering said magnetic particles to the interior side wall of said reaction receptacle, and
e) detecting said detection signal corresponding to an analyte concentration in the supernatant of said reaction receptacle in the presence of the sequestered magnetic particles,
wherein steps c) to e) are carried out in the same analytical processing unit.

13. The method of claim 12, wherein said analyte is selected from the group consisting of a nucleic acid and a polypeptide.

14. The method of claims 12, wherein processing of said nucleic acid comprises amplification.

15. The method according to claim 12, wherein said detection signal is an optical detection signal.

16. The method according to claim 15, wherein the optical detection signal is fluorescence.

17. The method according to claim 12, wherein said processing comprises elution of said substrate from the magnetic beads into the fluid in said reaction receptacle.

18. The method according to claim 13, wherein said processing of said polypeptide in step c) further comprises:
c1) contacting said analyte bound to magnetic particles with a detection molecule capable of binding said analyte, and
c2) eluting said detection molecule bound to said analyte to obtain a detection signal in a fluid in said reaction receptacles.

19. The method according to claim 18, wherein the detection molecule used in step c1) is a labeled antibody.

20. The method according to claim 12, wherein said magnetic field is produced by a magnetic assembly integrated into a thermoblock.
